(19) United States
(12) Patent Application Publication
(10) Pub. No.: US 2021/0192717 A1
(43) Pub. Date: Jun. 24, 2021

(54) SYSTEMS AND METHODS FOR IDENTIFYING ATHEROMATOUS PLAQUES IN MEDICAL IMAGES

(71) Applicant: Petuum Inc., Pittsburgh, PA (US)

(72) Inventors: Najmeh Sadoughi, Pittsburgh, PA (US); Suhaila Mumtaj Shakiah, Pittsburgh, PA (US); Pengtao Xie, Pittsburgh, PA (US); Eric Xing, Pittsburgh, PA (US)

(21) Appl. No.: 16/719,695

(22) Filed: Dec. 18, 2019

(51) Int. Cl. G06T 7/00; A61B 3/10; G06T 7/11

(52) U.S. Cl. CPC G06T 7/0012; A61B 3/102; G06T 7/11; G06T 2207/10072; G06T 2207/30101; G06T 2207/10016; G06T 2207/20084

(57) ABSTRACT

The current disclosure is directed towards providing systems and methods for identifying atheromatous plaques in optical coherence tomography (OCT) images. In one example, a method for a trained neural network may include acquiring an OCT image slice of an artery, identifying one or more image features of the OCT image slice with the trained neural network, and responsive to the one or more image features indicating a thin-cap fibroatheroma (TCFA), segmenting the OCT image slice into a plurality of regions with the trained neural network, the plurality of regions including a first region depicting the TCFA, and determining start and end coordinates for the TCFA based on the first region.

### FIELD

The present description relates generally to medical imaging, such as optical coherence tomographic imaging, and more particularly to systems and methods for identifying atheromatous plaques in optical coherence tomography images.

### BACKGROUND

Atheromatous plaques may build up in arteries, which, when left untreated, may result in thrombosis. Thrombosis may further lead to acute coronary syndromes detrimental to human health (e.g., sudden coronary death). Certain types of atheromatous plaques, such as thin-cap fibroatheromas (TCFAs), are of particular interest, as these plaques are vulnerable to potentially lethal plaque ruptures. It is therefore desirable to know where a TCFA is within the artery so that appropriate medical intervention (e.g., a surgical procedure) may be taken to prevent such ruptures.

To allow medical professionals to locate TCFAs, medical imaging techniques, such as optical coherence tomography (OCT), have been developed to image arteries. For example, OCT imaging techniques may scan at least a portion of the artery to generate a three-dimensional volumetric dataset from which two-dimensional cross-sections, or image slices, of the artery may be generated. The OCT image slices are often displayed in polar coordinates, in effect “unrolling” the wall of the artery (initially imaged in Cartesian coordinates as a circular shape) to facilitate TCFA identification. The medical professional may then mark a start coordinate and an end coordinate on a given image slice, indicating a location of the TCFA.

However, there are limitations to human identification. For example, differing medical professionals with varying levels of experience may identify different start and end coordinates for the TCFA (e.g., wider or tighter bounds), leading to inconsistent treatment. In some cases, the medical professional may accidentally miss a TCFA entirely, which may delay diagnosis. Advanced automated imaging techniques have been employed, but to limited success. Though such techniques may be able to detect inconsistencies between images, prior implementations have not been successful at determining locations for the TCFA in a replicable fashion. Thus, a challenge persists in the art to provide accurate, consistent, and automated identification of a location of a TCFA in OCT image slices.

The inventors have identified the above problems and herein provide systems and methods to at least partially address them.

### SUMMARY

The current disclosure provides systems and methods for training and using a neural network to identify atheromatous plaques in optical coherence tomography (OCT) images. In one example, a method for a trained neural network may include acquiring an OCT image slice of an artery, identifying one or more image features of the OCT image slice with the trained neural network, and responsive to the one or more image features indicating a thin-cap fibroatheroma (TCFA), segmenting the OCT image slice into a plurality of regions with the trained neural network, the plurality of regions including a first region depicting the TCFA, and determining start and end coordinates for the TCFA based on the first region.

In another example, a method may include training a neural network to identify a TCFA in OCT image slices, where identifying the TCFA may include identifying TCFA features in the OCT image slices, and generating bounding boxes in the OCT image slices for the TCFA based on the TCFA features, receiving a particular OCT image slice depicting a particular TCFA, and identifying the particular TCFA in the particular OCT image slice using the trained neural network.

In yet another example, a medical imaging system may include a scanner operable to collect OCT imaging data of a plaque, a memory storing a trained neural network configured to separate visual characteristics from content of an image, and a processor communicably coupled to the scanner and the memory, wherein the processor is configured to receive the OCT imaging data from the scanner, generate a sequentially ordered set of OCT images from the OCT imaging data, where a subset of the OCT images may depict the plaque, identify, via the trained neural network, the subset of OCT images depicting the plaque, generate, via the trained neural network, a bounding box circumscribing the plaque in each OCT image in the subset of OCT images, and determine, for each OCT image in the subset of OCT images, start and end coordinates for the plaque based on the bounding box.

The above examples may provide several advantages over the current state of the art. Typically, OCT images received for a given scan of a given patient may include ˜270 frames. A medical professional may necessarily resort to inspection of each individual frame to determine TCFA boundaries. Such a process may be both time-consuming and may invite human error and oversight. In contrast, the methods and systems provided herein may, in some examples, provide medical professionals with an assessment for the ˜270 frames in less than 10 seconds. As such, a given medical professional may save time during particularly time-sensitive medical procedures, such as intraoperative surgeries, where the medical professional may need to localize critical regions quickly. Further, since detection and localization of TCFAs in OCT images may be challenging and thus subject to differences in even experienced opinions, agreement between medical professionals may be low, varying from one medical professional to another.

According to at least some of the embodiments provided herein, a machine learning framework may mitigate such inaccuracies and disagreements by learning agreed TCFA boundaries from multiple medical professionals in a systematic manner. As such, medical professionals may be provided with a means for increasing precision of both medical procedures and of their personal skill level in TCFA detection, and thus improved agreements between medical professionals in diagnoses of TCFAs may be achieved.

It should be understood that the summary above is provided to introduce in simplified form a selection of concepts that are further described in the detailed description. It is not meant to identify key or essential features of the claimed subject matter, the scope of which is defined uniquely by the claims that follow the detailed description. Furthermore, the claimed subject matter is not limited to implementations that solve any disadvantages noted above or in any part of this disclosure.

### BRIEF DESCRIPTION OF THE DRAWINGS

FIG. 1 shows an example optical coherence tomography (OCT) imaging system.

FIG. 2 shows a high-level flow diagram of a processor operable to receive OCT imaging data as an input and output a thin-cap fibroatheroma (TCFA) region.

FIG. 3 shows a high-level flow diagram of a preprocessing module operable to preprocess the OCT imaging data.

FIG. 4 shows a schematic diagram illustrating an example neural network used for identifying a TCFA in an OCT image.

FIG. 5 shows a schematic diagram of an example process for generating bounding boxes on the OCT image using the example neural network.

FIG. 6A shows a schematic diagram of an overlap between two pairs of vertical lines superimposed on the OCT image.

FIG. 6B shows a schematic diagram of an overlap between two boxes superimposed on the OCT image.

FIG. 7 shows an example plot of an agreement between doctors in identification of OCT images depicting TCFAs.

FIG. 8 shows an example plot of an agreement between doctors in identification of TCFA regions in OCT images.

FIG. 9 shows a flow chart of a method for identifying the TCFA in the OCT image and displaying the OCT image.

FIG. 10 shows a flow chart of a first exemplary method for training the neural network to identify TCFAs in OCT images.

FIG. 11 shows a flow chart of a second exemplary method for training the neural network to identify TCFAs in OCT images.

### DETAILED DESCRIPTION

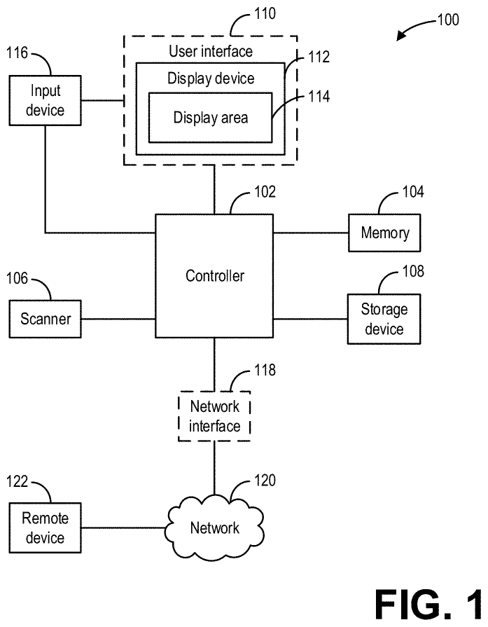

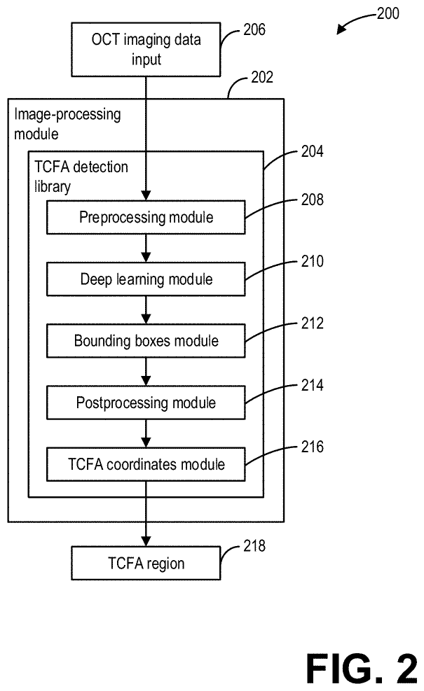

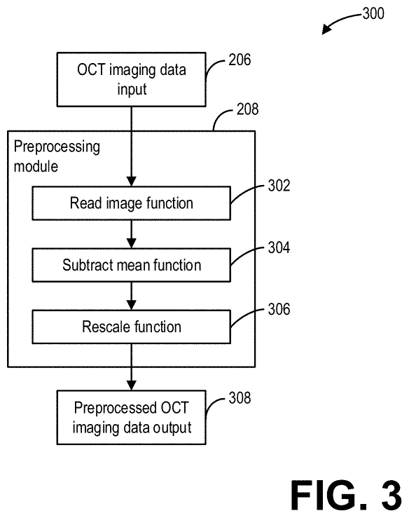

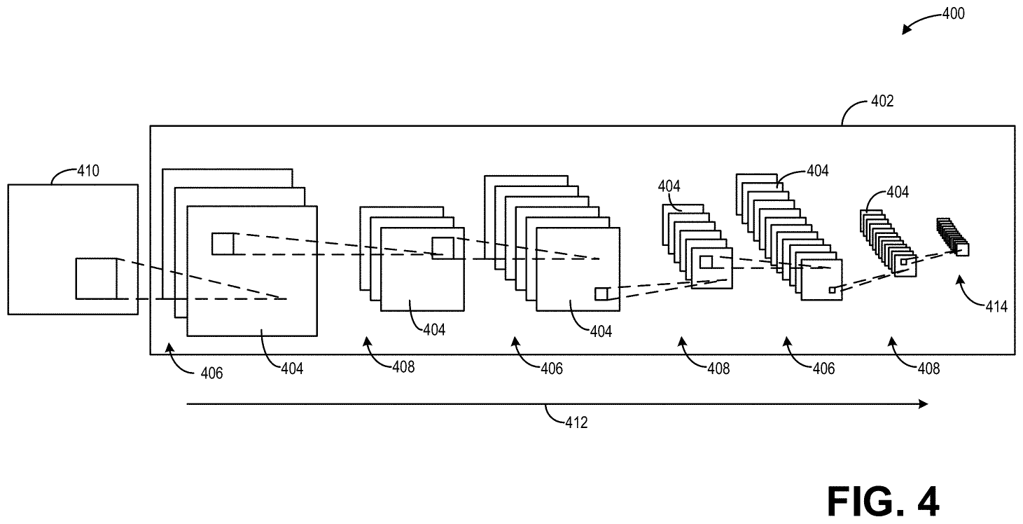

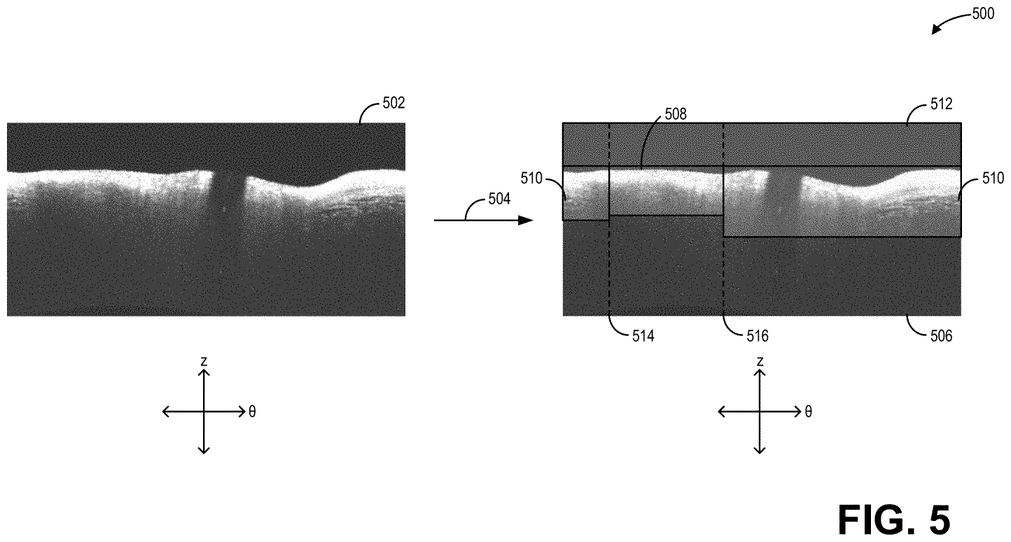

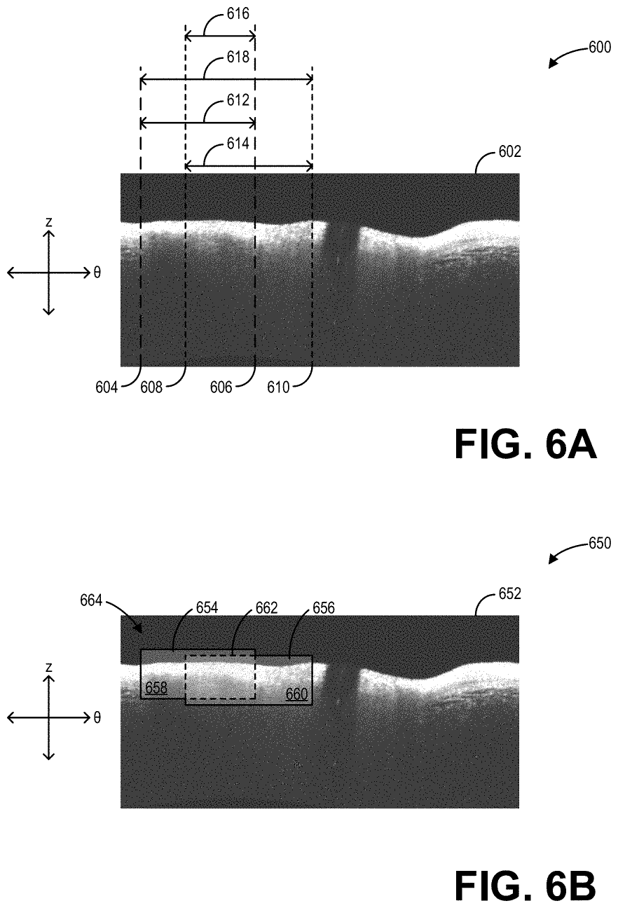

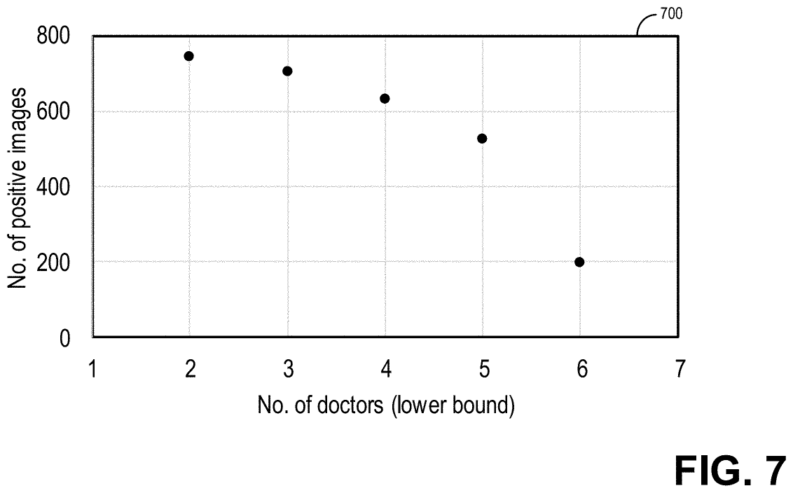

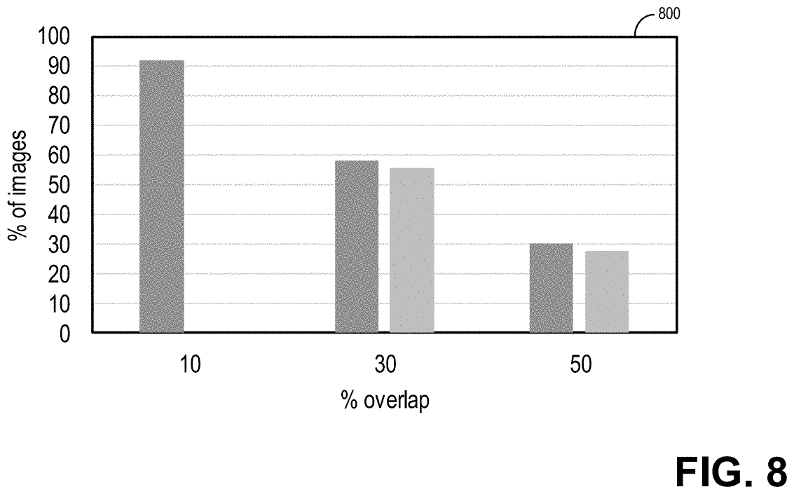

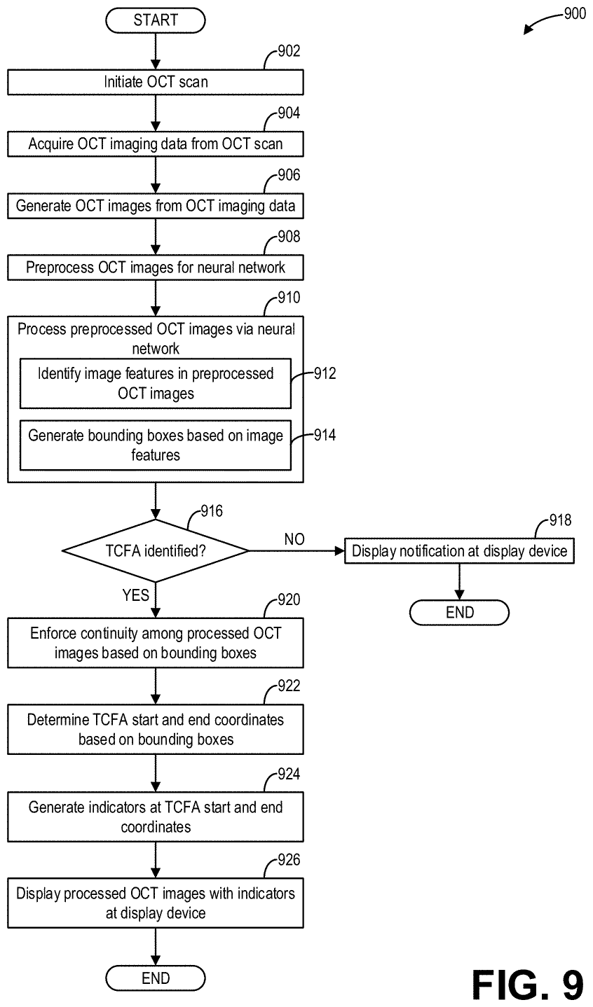

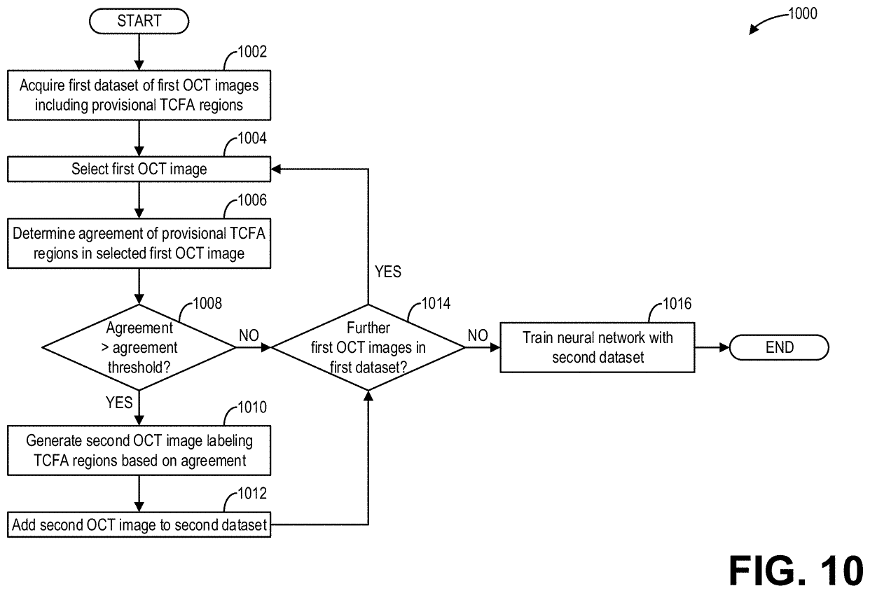

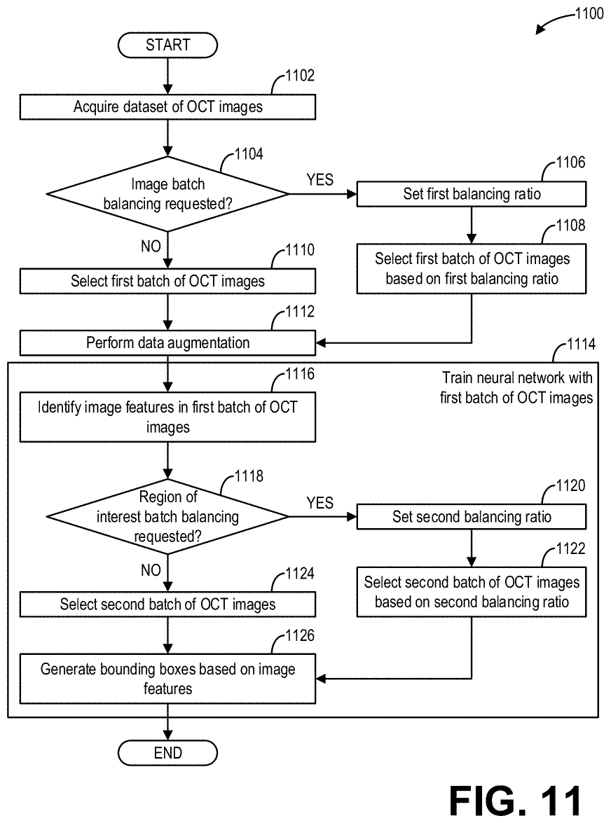

The current disclosure provides systems and methods for training and using a neural network to identify atheromatous plaques, such as thin-cap fibroatheromas (TCFAs), in medical images, such as optical coherence tomography (OCT) images. One example OCT imaging system for generating and displaying OCT images, and identifying TCFAs therein, is depicted at FIG. 1. FIG. 2 depicts a high-level flow diagram for a processor operable to read an OCT imaging data input and output a processed OCT image for display via the OCT imaging system of FIG. 1, for example. FIG. 3 depicts a high-level flow diagram for a preprocessing module stored on a processor, such as the processor of FIG. 2, where the preprocessing module may be operable to read the OCT imaging data input and output preprocess OCT imaging data. An example convolutional neural network (CNN) configured to separate visual characteristics from content of an image is depicted at FIG. 4. The example CNN may be implemented on the OCT imaging system of FIG. 1 to identify TCFAs in OCT images. An example process for generating bounding boxes with an example neural network, such as the example CNN of FIG. 4, is depicted at FIG. 5. FIGS. 6A and 6B depict two example overlaps between TCFA regions. Example plots showing agreement between doctors in TCFA identification are depicted at FIGS. 7 and 8. A method for identifying the TCFA in the OCT image and display the image is depicted at FIG. 9. Exemplary methods for training an example neural network, such as the example CNN of FIG. 4, are depicted at FIGS. 10 and 11.

Referring now to FIG. 1, a block diagram of an example system 100 is depicted according to an embodiment. In the illustrated embodiment, the system 100 is an imaging system and, more specifically, an OCT imaging system. However, it is understood that embodiments set forth herein may be implemented using other types of medical imaging modalities (e.g., magnetic resonance, ultrasound, etc.). Furthermore, it is understood that other embodiments do not actively acquire medical images. Instead, embodiments may retrieve image or OCT data that was previously acquired by an imaging system and analyze the image data as set forth herein. As shown, the system 100 includes multiple components. The components may be coupled to one another to form a single structure, may be separate but located within a common room, or may be remotely located with respect to one another. For example, one or more of the modules described herein may operate in a data server that has a distinct and remote location with respect to other components of the system 100, such as a probe and user interface. Optionally, in the case of OCT systems, the system 100 may be a unitary system that is capable of being moved (e.g., portably) from room to room. For example, the system 100 may include wheels or be transported on a cart.

In the illustrated embodiment, the system 100 may include a scanner 106 which may deliver continuous or pulsed low-coherence light into a body or volume (not shown) of a subject. The low-coherence light may be back-scattered from structures (e.g., an artery) in the body to produce echoes subsequently collected as OCT image signals. Scanners such as scanner 106 are well-known to those skilled in the art and will therefore be referenced only generally herein as relates to the described embodiments. The scanner 106 may be included in an intracoronary OCT probe attached to, or implemented in, a catheter, which may be utilized in a medical intervention procedure to image at least a portion of an artery of a subject. As such, the scanner 106 may be operable to collect OCT imaging data, such as three-dimensional (3D) volumetric OCT imaging data, depicting the artery. The artery may have a plaque, such as a TCFA, and an operator of the system 100 may utilize the scanner 106 to image the plaque. The 3D volumetric OCT imaging data may be processed as a set, or series, of sequential two-dimensional (2D) OCT image slices along a length of the artery, each of which may depict a cross section of a wall of the artery, for example. In some examples, an imaging resolution of less than 20 μm may be obtained by the scanner 106. The scanner 106 may be communicably coupled to a system controller 102 that may be part of a single processing unit, or processor, or distributed across multiple processing units. The system controller 102 is configured to control operation of the system 100.

For example, the system controller 102 may include an image-processing module (as described in greater detail below with reference to FIG. 2) that receives image data (e.g., OCT image signals) and processes the image data. For example, the image-processing module may process 3D volumetric OCT imaging data to generate 2D image slices of OCT information (e.g., OCT images) for displaying to an operator (not shown) of the system 100. Similarly, the image-processing module may process the OCT image signals to generate 3D renderings of OCT information (e.g., OCT images) for displaying to the operator. When the system 100 is an OCT system, the image-processing module may be configured to perform one or more processing operations according to a plurality of selectable OCT modalities on the acquired OCT information. For example, the image-processing module may implement a TCFA detection library for the identification of TCFAs in the OCT images.

Acquired OCT information may be processed in real-time during an imaging session (or scanning session) as the echoed signals are received. Additionally or alternatively, the OCT information may be stored temporarily in a memory 104 during an imaging session and processing less than real-time in a live or off-line operation. For longer-term storage, a storage device 108 is included for storing processed slices of acquired OCT information that are not scheduled to be displayed immediately. Further, the storage device 108 may store one or more datasets, such as training sets, for use with the image-processing module. The storage device 108 may include any known data storage medium, for example, a permanent storage medium, removable storage medium, and the like. Additionally, either or both of the memory 104 and the storage device 108 may be a non-transitory storage medium.

In operation, an OCT system may acquire data, for example, volumetric datasets, by various techniques (for example, 3D scanning, real-time 3D imaging, volume scanning, and the like). OCT images may be generated from the acquired data (at the controller 102) and displayed to the operator or user on a display device 112. Further, the system controller 102 may be communicably coupled to the display device 112 via a user interface 110 that enables an operator to control at least some operations of the system 100. The user interface 110 may include hardware, firmware, software, or a combination thereof that enables an individual (e.g., an operator) to directly or indirectly control operation of the system 100 and the various components thereof. As shown, the user interface 110 may include a display device 112 having a display area 114. In some embodiments, the user interface 110 may be operably connected to one or more user interface input devices 116, such as a physical keyboard, mouse, and/or touchpad. In one example, a touchpad may be configured to the system controller 102 and display area 114, such that when a user moves a finger/glove/stylus across a face of the touchpad, a cursor atop a displayed OCT image on the display device 112 may move in a corresponding manner.

In an exemplary embodiment, the display device 112 is a touch-sensitive display (e.g., touchscreen) which may detect a presence of a touch from the operator on the display area 114 and may also identify a location of the touch in the display area 114. The touch may be applied by, for example, at least one of an individual's hand, glove, stylus, or the like. As such, the touch-sensitive display may also be characterized as an input device that is configured to receive inputs from the operator (such as a request to adjust or update an orientation of a displayed image). The display device 112 may also communicate information from the controller 102 to the operator by displaying the information to the operator. The display device 112 and/or the user interface 110 may also communicate audibly. The display device 112 is configured to present information to the operator during or after the imaging or data acquiring session. The information presented may include OCT images (e.g., one or more 2D slices), graphical elements, measurement graphics of the displayed images, user-selectable elements, user settings, and other information (e.g., administrative information, personal information of the patient, and the like).

In addition to the image-processing module, the system controller 102 may also include one or more of a graphics module, an initialization module, a tracking module, and an analysis module. The image-processing module, the graphics module, the initialization module, the tracking module, and/or the analysis module may coordinate with one another to present information to the operator during and/or after the imaging session. For example, the image-processing module may be configured to display an acquired image on the display device 112, and the graphics module may be configured to display designated graphics along with the displayed image, such as selectable icons (e.g., image rotation icons) and measurement parameters (e.g., data) relating to the image. One or more of the controller 102, the memory 104, and the storage device 108 may include algorithms and one or more neural networks (e.g., a system of neural networks) stored within a memory of the controller for automatically recognizing one or more anatomical features depicted by a generated OCT image, such as a 2D slice, as described further below with reference to FIGS. 4, 9, and 10. In some examples, the controller may include a deep learning module which includes the one or more deep neural networks and instructions for performing the deep learning and feature recognition discussed herein. In some embodiments, the one or more deep neural networks may include a convolutional neural network (CNN) implementing a two-stage object detection algorithm. In some embodiments, the one or more deep neural networks may include a trained neural network configured to separate visual characteristics from content of an image.

A screen of the display area 114 of the display device 112 may be made up of a series of pixels which display the data acquired with the scanner 106. The acquired data includes one or more imaging parameters calculated for each pixel, or group of pixels (for example, a group of pixels assigned the same parameter value), of the display, where the one or more calculated image parameters includes one or more of an intensity, velocity (e.g., blood flow velocity), color flow velocity, texture, graininess, contractility, deformation, and rate of deformation value. The series of pixels may then make up the displayed image generated from the acquired OCT data.

The system 100 may be a medical OCT system used to acquire imaging data of a scanned object (e.g., an artery of a subject). The acquired image data may be used to generate one or more OCT images which may then be displayed via the display device 112 of the user interface 110. The one or more generated OCT images may include one or more 2D image slices, for example.

In some embodiments, the system controller 102 may be communicably coupled to a network 120 via a network interface 118. For example, the system controller 102 may communicate with and/or across the network 120 in a wired and/or wireless manner via the network interface 118. The network 120 may be a wireless telephone network, a wireless local area network, a wired local area network, a wireless wide area network, a wired wide area network, etc. In some embodiments, the network interface 118 may allow message to be sent and/or received to and/or from other devices, such as a remote device 122, via the network 120 (e.g., the public Internet). In some embodiments, the network 120 may be regarded as a private network connection to the remote device 122 and may include, for example, a virtual private network or an encryption or other security mechanism employed over the public Internet. The remote device 122 may be a computing device, such as a personal computing device (e.g., a laptop, tablet, smartphone, etc.), or another system similar to system 100 (e.g., another medical imaging system).

Referring now to FIG. 2, a high-level flow diagram 200 of an image-processing module 202 including a TCFA detection library 204 is depicted. The image-processing module 202 may be implemented on system controller 102 of system 100 of FIG. 1, for example. As such, the image-processing module 202 may receive an OCT imaging data input 206 to be processed by the TCFA detection library 204, which may return processed OCT image data including a TCFA region 218. The TCFA detection library 204 may include one or more modules for processing of the OCT imaging data input 206. In the exemplary embodiment depicted at FIG. 2, the TCFA detection library 204 may include a preprocessing module 208, a deep learning module 210, a bounding boxes module 212, a postprocessing module 214, and a TCFA coordinates module 216.

The image-processing module 202 may be configured to receive OCT imaging data (e.g., 3D volumetric imaging data) from a scanner (e.g., 106). From the OCT imaging data, the image-processing module 202 may generate a sequentially ordered set of OCT images (e.g., 2D image slices) from the OCT imaging data. The sequentially ordered set of OCT images may form the OCT imaging data input 206, which may be input into the TCFA detection library 204 for processing. Upon receipt, the TCFA detection library 204 may first pass the OCT imaging data input 206 to the preprocessing module 208, an embodiment of which is described below with reference to FIG. 3.

Referring now to FIG. 3, a high-level flow diagram 300 of the preprocessing module 208 is depicted. As discussed above with reference to FIG. 2, the preprocessing module 208 may be included in a TCFA detection library (e.g., 204) implemented in an image-processing module (e.g., 202). The processor may further be included in a system (e.g., 100) operable for medical imaging. The preprocessing module 208 may receive the OCT imaging data input 206 for preprocessing for input to another module (e.g., the deep learning module 210 as described below with reference to FIG. 2).

The OCT imaging data input 206 may first be passed to a read image function 302, which may parse imaging data in the OCT imaging data input 206 for preprocessing. For example, the parsed data may include a matrix of pixel intensity values representing one or more images included in the OCT imaging data input 206. The parsed data may then be passed to a subtract mean function 304. The subtract mean function 304 may be operable to receive parsed data corresponding to one or more images (e.g., OCT image slices) and determine and subtract a mean value for each pixel as a function of that pixel's location in each of the one or more images. In other embodiments, the subtract mean function 304 may be operable to determine and subtract a mean value for one or more pixel channels in the parsed data. An output of the subtract mean function 304 may thus be less sensitive to detection of background “noise” (e.g., objects of non-interest which exhibit relatively little change across the one or more images). For example, in a plurality of OCT images, the background noise may include low light areas, or healthy portions of an artery. However, objects of interest which appear in relatively few images (e.g., a TCFA) may become more prominent and therefore may be more easily detectable by object detection algorithms. The output of the subtract mean function 304 may then be passed to a rescale function 306, where a standard deviation of the mean-subtracted pixel intensity values may be determined, and then used to rescale the mean-subtracted pixel intensity values (e.g., each of the mean-subtracted pixel intensity values may be divided by the respective standard deviation). The rescale function 306 may then output the preprocessed OCT imaging data output, which may be passed to another module (e.g., the deep learning module 210 as described below with reference to FIG. 2).

Referring again to FIG. 2, preprocessed OCT imaging data from the preprocessing module 208 may be passed to the deep learning module 210 and from there to the bounding boxes module 212. The deep learning module 210 and the bounding boxes module 212 may interface with a neural network, such as a convolutional neural network, an exemplary embodiment of which is described below with reference to FIG. 4. The neural network may include a two-stage object detection algorithm, which may identify one or more image features (e.g., at the deep learning module 210) and then generate one or more bounding boxes based on the one or more identified image features (e.g., at the bounding boxes module 212). For example, the one or more image features may correspond to a TCFA depicted by a given OCT image, and a bounding box may be generated to circumscribe the TCFA. In some examples, the deep learning module 210 may further include functionalities to enforce rotational invariance of the OCT imaging data and to provide resilience to noise in the image without a need for data augmentation, and to convert preprocessed OCT imaging data from an image space to a coordinate space for postprocessing. Each of the three aforementioned functionalities are known generally in the art, and specific algorithmic details are therefore omitted here for brevity. Further, as used herein, “bounding box” may refer to any two-dimensional shape which circumscribes a given region of an OCT image, and is not limited to boxes, rectangles, squares, etc. except where otherwise indicated.

After the one or more bounding boxes have been generated based on the identified image features, output from the bounding boxes module 212 may be passed to the postprocessing module 214, whereby continuity may be enforced in the set of OCT image slices. Because the set of OCT image slices is sequentially ordered, a given TCFA will appear in a sequential subset of images. As such, the postprocessing module 214 may correct for spurious or medically less important results. For example, the postprocessing module 214 may base continuity enforcement on a medically relevant TCFA persisting in four to six sequential OCT image slices. In some examples, the four to six sequential OCT image slices may spatially correspond to ˜1 mm of an imaged artery.

As an example, the postprocessing module 214 may identify a first series of OCT image slices, the first series of OCT image slices including at least five sequential OCT image slices depicting the TCFA. If a TCFA is instead identified in less than five sequential OCT image slices, the postprocessing module 214 may indicate that no TCFA is present in these OCT image slices, as the TCFA may be spuriously identified, or may be small enough that the TCFA may heal on its own (e.g., without medical intervention).

As another example, the postprocessing module 214 may identify a second series of OCT image slices, the second series of OCT image slices including at least five sequential OCT images, wherein at least a first OCT image slice and a last OCT image slice in the second series is identified as including the TCFA, and only one remaining OCT image in between the first OCT image slice and the last OCT image slice is identified as not including the TCFA. The postprocessing module 214 may enforce continuity by indicating the TCFA in each of the OCT image slices in the second series, including the one remaining OCT image slice.

As yet another example, the postprocessing module 214 may identify a third series of OCT image slices, the third series of OCT image slices including a sequential ordering of a first OCT image slice, a second OCT image slice, and a third OCT image slice, where the second OCT image slice is identified as including the TCFA, and the first OCT image slice and the third OCT image slice are identified as not including the TCFA. The postprocessing module 214 may enforce continuity by indicating no TCFA in the second OCT image slice, as the initially identified TCFA in the second OCT image slice may be interpreted by the postprocessing module 214 as spuriously detected by the neural network.

After continuity has been enforced by the postprocessing module 214, the OCT image slices output to the TCFA coordinates module 216. The TCFA coordinates module 216 may be operable to generate start and end coordinates for the TCFA based on the bounding box generated to circumscribe the TCFA. In some examples, the start and end coordinates may be polar spatial coordinates. Visual indicators may be respectively generated on the start and end coordinates of each OCT image slice determined to depict the TCFA. After being output from the TCFA detection library as the processed OCT imaging data including the TCFA region 218, a subset of the OCT image slices depicting the TCFA and including the visual indicators may be displayed at a display area (e.g., 114) of a display device (e.g., 112) to a medical professional (e.g., an operator of the system 100).

Referring now to FIG. 4, a schematic diagram 400 of an example neural network 402 used for object detection and identification in image inputs (e.g., detection and identification of TCFAs in OCT image slices) and generating bounding boxes therefor is depicted. The neural network 402 may be included in a controller of an imaging system (e.g., controller 102 of system 100 of FIG. 1) and/or in a system in electronic communication with the controller of the imaging system (or receiving data from the controller of the imaging system). The neural network 402 may be a convolutional neural network 402. Convolutional neural networks are a class of biologically inspired deep neural networks that are powerful in image processing tasks. In particular, convolutional neural networks are modeled after the visual system of the brain. Unlike a “traditional” neural network, convolutional neural networks consist of layers organized in three dimensions and neurons in one layer are connected to only a subset of neurons in the next layer (instead of connecting to all neurons, such as in densely connected layers).

As shown in FIG. 4, the convolutional neural network 402 may consist of layers of computational units that process visual information hierarchically in a feed-forward manner. The output of each layer may include a plurality of feature maps 404 which may be understood as differently filtered versions of an input image 410. For example, the convolutional neural network 402 may include a plurality of convolutional layers 406 and pooling layers 408. Though the convolutional layers 406 and pooling layers 408 are shown in an alternating pattern in FIG. 4, in some embodiments, there may be more or less convolutional layers and/or more or less pooling layers and the number of convolutional layers and pooling layers may not be equal and may not be in the alternating pattern. The input image 410 (e.g., a preprocessed OCT image slice) may be input into the convolutional neural network 402. The input image 410, and each image of the feature maps 404, may be represented as a matrix of pixel intensity values. The matrices of pixel intensity values may be understood as the data which may be used by the convolutional neural network 402. Though a single input image 410 is shown in FIG. 4, as described herein, a plurality of sequential input images may be input into the convolutional neural network 402.

Convolution may occur at each of the convolutional layers 406. Convolution may be performed in order to extract features from the input image 410 (or the feature maps 404 in higher layers further along in the processing hierarchy). Convolution preserves the spatial relationship between pixels by mapping image features from a portion of a first layer to a portion of a second layer, using learning filters including a plurality of weights. Each convolutional layer 406 may include a collection of image filters, each of which extracts a certain feature from the given input image (e.g., 404, 410). The output of each convolutional layer 406 may include a plurality of feature maps 404, each being a differently filtered version of the input image. In some examples, there may be one resulting feature map 404 per applied filter.

Pooling (e.g., spatial pooling, which may be max pooling in one example) may occur at each of the pooling layers 408. Pooling may be performed in order to reduce a dimensionality (e.g., size) of each feature map 404 while retaining or increasing certainty of feature identification. By pooling, a number of parameters and computations in the neural network 402 may be reduced, thereby controlling for overfitting, and a certainty of feature identification may be increased.

As shown in FIG. 4, following the first convolution, three feature maps 404 may be produced (however, it should be noted that this number may be representative and there may be greater than three feature maps in the first convolutional layer 406). Following the first pooling operation, the size of each feature map 404 may be reduced, though the number of feature maps 404 may be preserved. Then, during the second convolution, a larger number of filters may be applied and the output may be a correspondingly greater number of feature maps 404 in the second convolutional layer 406. Later layers along the processing hierarchy, shown by directional arrow 412, may be referred to as “higher” layers. The first few layers of the processing hierarchy may detect larger features while the later (higher) layers may pick up finer details and organize such details into more complex features. In some embodiments, a final output layer 414 may be fully connected (e.g., all neurons in the final output layer 414 may be connected to all neurons in the previous layer). However, in other embodiments, final output layer 414 may not be fully connected.

By training the convolutional neural network 402 on object recognition, the convolutional neural network 402 may develop a representation of the input image 410 which makes object information increasingly explicit along the processing hierarchy (as shown by arrow 412). Thus, along the processing hierarchy of the convolutional neural network 402, the input image 410 may be transformed into representations which increasingly emphasize the actual content of the input image 410 compared to its detailed pixel intensity values. Images reconstructed from the feature maps 404 of the higher layers in the convolutional neural network 402 may capture the high-level content in terms of objects and their arrangement in the input image 410 but may not constrain exact pixel intensity values of the content reconstructions. In contrast, image reconstructions from the lower layers may reproduce the exact pixel intensity values of the original input image 410. Thus, feature responses in the higher (e.g., deeper) layers of the convolutional neural network 402 may be referred to as the content representation.

In an exemplary embodiment, the convolutional neural network 402 may be employed to identify one or more image features corresponding to a TCFA in an imaged artery. The input image 410 may therefore include one or more preprocessed OCT image slices depicting the artery having the TCFA. The one or more preprocessed OCT image slices may be scaled by a preprocessing module (e.g., 208) such that the convolutional neural network 402 may be more easily able to distinguish subtle variations between regions of the images and thereby identify the one or more image features. Upon identification of the one or more image features (e.g., the high-level content) in a given OCT image slice, outputted objects may be used as inputs for segmentation of the OCT image slice into one or more regions. As will be further discussed below with reference to FIG. 5, the one or more regions may be respectively bound by one or more bounding boxes generated based on the one or more image features, wherein at least one of the one or more bounding boxes circumscribes the depicted TCFA. As such, the convolutional neural network 402 may be considered a two-stage object detection algorithm which identifies one or more TCFAs depicted by an inputted OCT image slice and then generates one or more bounding boxes based on the one or more identified TCFAs.

Referring now to FIG. 5, a schematic diagram 500 of an example process 504 for generating bounding boxes and TCFA coordinates on an OCT image using a neural network is depicted. The neural network may be the convolutional neural network 402 of FIG. 4, for example. A first image 502 may form an input for the example process 504, which may be processed to obtain a second image 506. Each of the first image 502 and the processed second image 506 may be an OCT image slice of an artery having a TCFA. Further, each of the first image 502 and the second image 506 may depict the artery in polar spatial coordinates (as indicated by the z- and ( ) axes), such that a wall of the artery is depicted as “unrolled” in each image (as opposed to circular, such as when the artery is depicted in Cartesian spatial coordinates). Depicting OCT image slices in polar coordinates may help medical professionals viewing the OCT image slices to make diagnoses.

The second image 506 may include one or more rectangular bounding boxes generated via the example process 504 corresponding to one or more regions of the second image 506. In general, the neural network employed by the example process 504 may divide a given image into three parts: one or more healthy portions of the artery, one or more unhealthy portions of the artery (e.g., TCFAs), and a low-light background. As shown in FIG. 5, a first bounding box 508 may circumscribe a first region depicting the TCFA, such that the first region may bounded by the first bounding box 508. In some examples not depicted by FIG. 5, a plurality of first bounding boxes 508 may be generated, each of which may circumscribe an additional first region depicting an additional TCFA. As further shown by FIG. 5, one or more second bounding boxes 510 may circumscribe one or more second regions respectively depicting one or more healthy portions of the artery, such that each second region may be bounded by one of the one or more second bounding boxes 510. Further, one or more third bounding boxes 512 may circumscribe one or more third regions respectively corresponding to the low-light, or black, background not containing visually discernable structures, such that each third region may be bounded by one of the one or more third bounding boxes 512. The one or more bounding boxes may not include the entire second image 506, as at least some of the second image 506 may not be cleanly categorized as a healthy portion of the artery, an unhealthy portion of the artery, or low-light background. As such, the one or more bounding boxes may not extend an entire length of the second image 506 along the z-axis.

The example process 504 may further determine a start coordinate and an end coordinate for the second image 506. Specifically, the example process 504 may determine coordinates along two opposite sides of the first bounding box 508 parallel to the z-axis. Thus, the two opposite sides of the first bounding box 508 may be approximately perpendicular to a length of the depicted TCFA in polar coordinates. As used with reference to FIG. 5, “approximately” may refer to within 10° of orthogonality between a line approximating the length of the depicted TCFA in polar coordinates and the two sides of the first bounding box 508 parallel to the z-axis. A first dashed line 514 and a second dashed line 516, each parallel to the z-axis, are superimposed on the second image 506 in FIG. 5 to indicate the start and end coordinates, respectively. In this way, the neural network of the present disclosure may segment an OCT image slice into a plurality of regions, the plurality of regions including a first region depicting a TCFA, and may further determine start and end coordinates for the TCFA based on the first region.

Referring now to FIGS. 6A-8, a training dataset for the neural network (e.g., the convolutional neural network 402 of FIG. 4) may be developed by first determining an overlap of a plurality of provisional TCFA regions in OCT images of an initial dataset. Two example processes for determining this overlap are respectively depicted by FIGS. 6A and 6B. The plurality of provisional TCFA regions may be respectively selected by a plurality of medical professionals, for example. Two plots depict features of an example initial dataset of OCT images including TCFA regions selected by medical professionals are respectively depicted by FIGS. 7 and 8. As used herein, “provisional TCFA regions” may refer to TCFA regions present in the initial dataset prior to generation of the overlap regions for the training dataset; thus, the plurality of provisional TCFA regions may not directly be used in training of the neural network.

In some examples, the neural network may have difficulty visually recognizing a TCFA region without feedback from a plurality of medical professionals. Specifically, TCFA regions may be particularly difficult to recognize visually without experienced guidance to indicate which portions of a given image correspond to TCFA regions. However, the neural network may not be adequately trained based on feedback from one medical professional alone, as any given medical professional may bias results of the training. Thus, the neural network may be trained using a training dataset of images including composite overlapped regions corresponding to agreement by a plurality of medical professionals. These composite overlapped regions may serve as the “ground truth” TCFA regions during training of the neural network. Further, the neural network may be trained to assign higher weights to overlapped regions which correspond to agreement by a greater number of medical professionals. In this way, the neural network may aggregate the combined experience of the plurality of medical professionals and learn which image features are typically agreed upon as corresponding to TCFA regions.

Referring now to FIG. 6A, a schematic diagram 600 of an overlap between two pairs of vertical lines superimposed on an OCT image 602 is depicted. The OCT image 602 may depict an artery having a TCFA in polar spatial coordinates (as indicated by the z- and θ-axes). A first long-dashed line 604 and a second long-dashed line 606 may indicate where a first medical professional has identified a first provisional TCFA region. A first short-dashed line 608 and a second short-dashed line 610 may indicate where a second medical professional has identified a second provisional TCFA region. Further, each of the lines 604, 606, 608, 610 may be parallel to the z-axis.

The pair of long-dashed lines 604, 606 and the pair of short-dashed lines 608, 610 may respectively define a first length 612 and a second length 614. The first length 612 may correspond to the first provisional TCFA region and the second length 614 may correspond to the second provisional TCFA region. The overlap between the first and second provisional TCFA regions may be determined as a ratio of the intersection 616 of the first length 612 and the second length 614 to the union 618 of the first length 612 and the second length 614. As such, the overlap exemplified by the schematic diagram 600 may be regarded as a one-dimensional intersection over union (IoU) metric. As a value of the overlap grows higher, a training algorithm may assign a greater confidence (e.g., weight) to the overlap as corresponding to a ground truth TCFA region.

Referring now to FIG. 6B, a schematic diagram 650 of an overlap between two boxes superimposed on an OCT image 652 is depicted. The OCT image 652 may depict an artery having a TCFA in polar spatial coordinates (as indicated by the z- and θ-axes). A first box 654 may indicate where a first medical professional has identified a first provisional TCFA region. A second box 656 may indicate where a second medical professional has identified a second provisional TCFA region. Each of the first box 654 and the second box 656 may be respectively oriented such that two sides are parallel to the z-axis and two sides are parallel to the θ-axis.

The first box 654 and the second box 656 may respectively define a first area 658 and a second area 660. The first area 658 may correspond to the first provisional TCFA region and the second area 660 may correspond to the second provisional TCFA region. The overlap between the first and second provisional TCFA regions may be determined as a ratio of the intersection 662 (encompassed by dashed lines in FIG. 6B) of the first area 658 and the second area 660 to the union 664 (encompassed by solid lines in FIG. 6B) of the first area 658 and the second area 660. As a value of the overlap grows higher, a training algorithm may assign a greater confidence (e.g., weight) to the overlap as corresponding to a ground truth TCFA region.

Referring now to FIG. 7, an example plot 700 is depicted, the example plot 700 showing an agreement between medical professionals (e.g., doctors) in identification of OCT images depicting TCFAs. Plotted along an ordinate is a total number of positive images, where a “positive image” may refer to an OCT image indicated by a doctor as depicting a TCFA (as opposed to a “negative image,” which may refer to an OCT image indicated by a doctor as not depicting a TCFA). Plotted along an abscissa is a number of doctors that agree that a particular image is a positive image.

The dataset depicted by the example plot 700 includes 5684 OCT images corresponding to OCT imaging data for 21 patients, where 1000 OCT images are identified as positive images by at least one doctor in a group of six doctors. As shown by the example plot 700, of the 1000 positive images, at least two doctors identify a TCFA in 745 images, at least three doctors identify a TCFA in 706 images, at least four doctors identify a TCFA in 633 images, at least five doctors identify a TCFA in 525 images, and all six doctors identify a TCFA in 197 images. In one example, the ground truth for the training dataset may be selected as the 197 images of which all six doctors agree depict a TCFA.

Referring now to FIG. 8, an example plot 800 is depicted, the example plot 800 showing an agreement between medical professionals (e.g., doctors) in identification of provisional TCFA regions in OCT images. Plotted along an abscissa is a percentage of overlap between provisional TCFA regions in a given set of OCT images, where the percentage of overlap may be determined via one of the processes described with reference to FIGS. 6A and 6B. Plotted along an ordinate is a percentage of images in a given set of OCT images which have at least the percentage of overlap plotted by the abscissa. The darker bars plot results from the set of 745 images in which at least two doctors identified a TCFA, as described above with reference to FIG. 7. The lighter bars plot results from the set of 633 images in which at least four doctors identified a TCFA, as described above with reference to FIG. 7.

The dataset depicted by the example plot 800 may be the same dataset as that depicted by the example plot 700. As shown by the example plot 800, at least 10% overlap between provisional TCFA regions is present in 92% of the 745 images indicated as positive by at least two doctors, at least 30% overlap is present in 58% of these images, and at least 50% overlap is present in at least 30% of these images. Further, at least 30% overlap between provisional TCFA regions is present in 55.6% of the images indicated as positive by at last four doctors and at least 50% overlap is present in 27.6% of these images. In one example, the ground truth for the training dataset may be selected as the images agreed on by at least four doctors which exhibit at least 50% overlap between provisional TCFA regions.

Referring now to FIG. 9, a flow chart of a method 900 for identifying a TCFA in an OCT image and displaying the OCT image is depicted. Method 900 will be described with reference to the embodiments provided hereinabove, though it may be understood that similar methods may be applied to other systems without departing from the scope of this disclosure. For example, method 900 may be executed by the system 100 of FIG. 1. Specifically, method 900 may be carried out via the controller 102, and may be stored as executable instructions at a non-transitory storage medium, such as the memory 104 or the storage device 108.

At 902, method 900 may include initiating an OCT scan. The OCT scan may be performed with a scanner (e.g., 106) which emits, and collects echoes from, low-coherence light to image one or more anatomical structures of a subject. In some examples, the low-coherence light may be echoed from an artery having a plaque, such as a TCFA.

At 904, method 900 may include acquiring OCT imaging data. The low-coherence light collected by the scanner (e.g., 106) may correspond to OCT imaging data (e.g., 3D volumetric imaging data) depicting the artery having the TCFA. The OCT imaging data may be received by a controller (e.g., 102) may be communicably coupled to the scanner.

At 906, method 900 may include generating one or more OCT images (e.g., 2D image slices) from the OCT imaging data. The one or more OCT images may include a sequentially ordered series, or set, of OCT images. In some examples, the set of OCT images may include ˜270 image slices of a single OCT scan for one patient. In some examples, a single OCT image may be generated and subsequently processed at any one time. In other examples, each OCT image in the set of OCT images may be generated and subsequently processed one at a time. In other examples, each OCT image in the set of OCT images may be subsequently processed in parallel. Within the set of OCT images, a subset of OCT images may depict the plaque being imaged.

At 908, method 900 may include preprocessing the one or more OCT images for processing by a neural network. The preprocessing may occur via a preprocessing module, such as preprocessing module 208, as described above with reference to FIGS. 2 and 3. The preprocessing may include preparing data encoding the one or more OCT images for the neural network. For example, the one or more OCT images may be averaged and normalized so that the neural network may distinguish between subtle regions of each OCT image and extract one or more TCFA regions therein.

At 910, method 900 may include processing the one or more preprocessed OCT images via the neural network. The neural network may be configured to separate visual characteristics from content of an image. As such, the neural network may be a convolutional neural network, such as the convolutional neural network 402 of FIG. 4. The neural network may include a trained two-stage object detection algorithm, which may identify one or more image features and then generate one or more bounding boxes based on the one or more image features.

Specifically, at 912, the neural network may identify one or more image features in one or more OCT images (e.g., the one or more preprocessed OCT images). The identifying may occur via a deep learning module, such as deep learning module 210, as described above with reference to FIG. 2. In some examples, the one or more image features may include one or more TCFA features (e.g., image features corresponding to one or more TCFAs). However, in some examples, some of the OCT images may not depict one or more TCFAs. For example, the neural network may identify one or more image features indicating a TCFA in a subset of OCT images, where all other remaining OCT images (e.g., apart from the subset) are identified as including no TCFA. In some examples, the neural network may identify one or more additional image features depicting one or more additional TCFAs in the subset of OCT images. In one example, the neural network may identify one or more additional image features depicting one or more additional TCFAs in a single OCT image. In this way, the neural network may identify one or more particular TCFAs in one or more particular OCT images after being trained to generally identify TCFAs in OCT images. In other examples, none of the OCT images may depict the TCFA, and the neural network may identify no TCFA in any of the OCT images.

Then, at 914, one or more bounding boxes may be generated based on the one or more image features. The generating may occur via a bounding boxes module, such as bounding boxes module 212, as described above with reference to FIG. 2. The neural network may segment each of the one or more OCT images into one or more regions based on the one or more image features. In examples wherein a TCFA is identified in the one or more OCT images (e.g., the subset of OCT images depicting the TCFA), the one or more regions may correspondingly include one or more first regions each depicting the TCFA. In examples wherein one or more additional TCFAs are identified in the one or more OCT images (e.g., the subset of OCT images depicting the TCFA), the one or more regions may include one or more second regions depicting the one or more additional TCFAs. In examples wherein one or more healthy portions of the artery are identified in the one or more OCT images, the one or more regions may correspondingly include one or more third regions depicting the one or more healthy portions of the artery. In examples wherein one or more low-light backgrounds are identified in the one or more OCT images, the one or more regions may correspondingly include one or more fourth regions depicting the one or more low-light backgrounds. Once the one or more OCT images is segmented into the one or more regions, the one or more bounding boxes may be generated based on the one or more regions. As such, the one or more regions may be respectively bound by the one or more bounding boxes. Thus, for example, a bounding box may be generated based on one of the one or more first regions such that the bounding box may circumscribe the TCFA indicated by the one or more TCFA features. In this way, the neural network may identify a TCFA and then localize the TCFA within an OCT image by generating a bounding box to circumscribe the TCFA.

At 916, method 900 may include determining whether the neural network has identified one or more TCFAs in the one or more OCT images. If no TCFA is identified by the neural network (e.g., if no image feature indicates a TCFA), method 900 may proceed to 918 to display a notification at a display area (e.g., 114) of a display device (e.g., 112). The notification may indicate to an operator of the system (e.g., 100) that no TCFA was identified during the OCT scan. In some examples, the display device may further be operable to display the one or more OCT images processed by the neural network at the display area. Method 900 may then end.

If one or more TCFAs are identified by the neural network (e.g., if one or more image features indicate a TCFA), method 900 may proceed to 920 to enforce continuity among the subset of processed OCT images (e.g., the OCT images identified by the neural network as including a TCFA) based on the one or more bounding boxes. The enforcing may occur via a postprocessing module, such as postprocessing module 214, as described above with reference to FIG. 2. As such, enforcing continuity may include one or more of the example operations described above with reference to FIG. 2, which will not be repeated here for brevity. That is, enforcing continuity may be based upon a medically relevant TCFA persisting in four to six sequential OCT image slices. Thus, at 920, method 900 may correct for spurious TCFAs (e.g., TCFAs erroneously identified by the neural network) or medically less important TCFAs (e.g., TCFAs which will heal on their own).

At 922, method 900 may include determining TCFA start and end coordinates in the subset of processed OCT images (e.g., the OCT images identified by the neural network as including a TCFA) based on the one or more bounding boxes. The determining may occur via a TCFA coordinates module, such as TCFA coordinates module 216, as described above with reference to FIG. 2. For a given TCFA in a given OCT image, the TCFA start and end coordinates may be determined based on a given bounding box circumscribing the TCFA (and thus, based on a given first region identified by the neural network as depicting the TCFA). Further, as the OCT images may be generated and processed in polar spatial coordinates, the TCFA start and end coordinates may correspondingly be polar spatial coordinates. For example, the TCFA start and end coordinates may be respectively determined as corresponding to two opposite sides of the given (rectangular) bounding box, where the two opposite sides of the given bounding box may be perpendicular, or approximately perpendicular, to a length of the given TCFA in polar coordinates.

At 924, method 900 may include generating visual indicators at each of the determined TCFA start and end coordinates on each of the subset of processed OCT images. The generating may also occur via the TCFA coordinates module (e.g., 216). The visual indicators may assist a medical professional (e.g., an operator of the system 100) in diagnosing a TCFA by indicating to the medical professional a location of the TCFA within the artery.

At 926, method 900 may include displaying the subset of processed OCT images with the generated visual indicators at the display area (e.g., 114) of the display device (e.g., 112). Thus, an operator of the system (e.g., 100) may be automatically presented with a clearly indicated depictions of any identified TCFAs in the artery of a subject. In some examples, the display device may further be operable to display one or more remaining processed OCT images (e.g., the OCT images indicated by the neural network as not depicting a TCFA) at the display area. Method 900 may then end.

Referring now to FIG. 10, a flow chart of a first exemplary method 1000 for training a neural network to identify TCFAs is depicted. Method 1000 will be described with reference to the embodiments provided hereinabove, though it may be understood that similar methods may be applied to other systems without departing from the scope of this disclosure. For example, method 1000 may be executed by the system 100 of FIG. 1 to train a neural network, such as the convolutional neural network 402 of FIG. 4. Specifically, method 1000 may be carried out via the controller 102, and may be stored as executable instructions at a non-transitory storage medium, such as the memory 104 or the storage device 108.

At 1002, method 1000 may include acquiring a first dataset of first OCT images (e.g., 2D image slices) for training the neural network, each of the first OCT images including one or more provisional TCFA regions. Each of the first OCT images may depict an artery of a subject, the artery having one or more TCFAs. Each TCFA depicted by each of the first OCT images may correspond to at least one of the one or more provisional TCFA regions. Each of the one or more provisional TCFA regions may be respectively received from one or more medical professionals. As such, the first dataset may be the dataset described with reference to FIGS. 7 and 8, for example. Thus, each provisional TCFA region may be judged by one of the one or more medical professionals to include a TCFA.

At 1004, method 1000 may include selecting one of the one or more first OCT images. In examples wherein the one or more first OCT images are in a sequential order, the selecting may also be performed in the sequential order.

At 1006, method 1000 may include determining an agreement of the one or more provisional TCFA regions in the selected first OCT image. Each of the one or more provisional TCFA regions may be partitioned into columns, each of which may be evaluated for a degree to which the one or more medical professionals who selected the one or more provisional TCFA regions agree with one another. In some examples, the agreement of the one or more medical professionals for each of the columns may be determined based on an amount of overlap. In such examples, the amount of overlap may be a percentage of overlap determined via one of the processes described above with reference to FIGS. 6A and 6B, respectively. The amount of overlap may represent a degree to which the one or more medical professionals who selected the one or more provisional TCFA regions agree with one another.

At 1008, method 1000 may include determining whether the agreement in the selected first OCT image is greater than an agreement threshold. The agreement threshold may be optimized to filter out TCFA regions which are less visually identifiable from training of the neural network. In some examples, the agreement threshold may be based on a majority agreement as to whether a respective TCFA is depicted by the columns of the one or more provisional TCFA regions (e.g., whether a majority of columns is determined to depict a portion of a TCFA).

If the agreement is greater than the agreement threshold, method 1000 may proceed to 1010 to generate a second OCT image indicating the agreement. The second OCT image may be generated from the same OCT imaging data as the selected first OCT image, and may only differ in one or more generated labels annotating a TCFA and/or a generated box indicating the amount of overlap; however, the one or more provisional TCFA regions may no longer be indicated in the second OCT image.

At 1012, method 1000 may include adding the generated second OCT image to a second dataset. The second dataset may be utilized to effectively train the neural network, as the second dataset will be less biased by an individual judgment of any one of the one or more medical professionals.

Once the second OCT image has been added to the second dataset, or if the agreement is less than the agreement threshold, method 1000 may proceed to 1014 to determine whether further first OCT images are in the first dataset. If further first OCT images are in the first dataset (e.g., for which the amount of overlap has not yet been determined), method 1000 may return to 1004 to select another first OCT image.

If an amount of overlap has been determined for each first OCT image in the first dataset, method 1000 may proceed to 1016 to train the neural network based on the second dataset. As an example, the neural network may be trained as at 1114 of method 1100, as described below with reference to FIG. 11. In this way, training of the neural network may be based upon the acquired first dataset of first OCT images which indicate medical opinions of one or more medical professionals as to a presence of a given TCFA. As such, the neural network may be trained to identify a TCFA in OCT images by identifying one or more TCFA features in the OCT images and then generating one or more bounding boxes in the OCT images for the TCFA based on the one or more TCFA features. Method 1000 may then end.

Referring now to FIG. 11, a flow chart of a second exemplary method 1100 for training a neural network to identify TCFAs is depicted. Method 1100 will be described with reference to the embodiments provided hereinabove, though it may be understood that similar methods may be applied to other systems without departing from the scope of this disclosure. For example, method 1100 may be executed by the system 100 of FIG. 1 to train a neural network, such as the convolutional neural network 402 of FIG. 4. Specifically, method 1100 may be carried out via the controller 102, and may be stored as executable instructions at a non-transitory storage medium, such as the memory 104 or the storage device 108.

At 1102, method 1100 may include acquiring a dataset of OCT images (e.g., 2D image slices) for training the neural network, each of the OCT images depicting an artery of a subject. The dataset of OCT images may include both positive and negative images, where the positive and negative images have been generated and labeled during a data preparation process. As such, in the positive images, the depicted artery may have one or more TCFAs, and in the negative images, the depicted artery may have no TCFA. Each TCFA depicted by each of the positive images may correspond to at least one of one or more provisional TCFA regions labeled on the positive image. Each of the one or more provisional TCFA regions may be respectively received from one or more medical professionals. As such, the positive images may correspond to the dataset described with reference to FIGS. 7 and 8, for example. Thus, each provisional TCFA region may be judged by one of the one or more medical professionals to include a TCFA.

At 1104, method 1100 may include determining whether an image batch balancing routine is requested. In general, batch balancing may be implemented to more uniformly capture features of dominant and minority classes. As an example, the positive images may be considered the dominant class and the negative images may be considered the minority class. If the acquired dataset is imbalanced, features of the dominant class may be overrepresented, for example. In the case of the method of the present disclosure, such imbalance may result in spurious predictions of TCFA regions where none are present in a given OCT image.

Provided within method 1100 are two exemplary instances where a batch balancing routine may be requested: during preprocessing of the OCT images for neural network training (e.g., at 1104) and during neural network training itself (e.g., at 1118). If the image batch balancing routine (e.g., during preprocessing) is requested, method 1100 may proceed to 1106 to set a first balancing ratio. The first balancing ratio may be a desired ratio of dominant class members to minority class members, that is, of positive images to negative images, for data preprocessing. The first balancing ratio may be predetermined to minimize spurious predictions of TCFA regions (or to minimize instances of missing TCFA regions). In one example, the first balancing ratio may be 90:10. In another example, the first balancing ratio may be 80:20. In yet another example, the first balancing ratio may be 70:30. In yet another example, the first balancing ratio may be 60:40. In yet another example, the first balancing ratio may be 50:50.

At 1108, method 1100 may include selecting a first batch, or image batch, of OCT images based on the first balancing ratio. For example, if the dataset includes at least 100 OCT images, and the first balancing ratio is set at 90:10, then method 1100 may include selecting 90 positive images and 10 negative images for neural network training. The selection may be performed arbitrarily, so as not to bias neural network training.

Returning to 1104, if the image batch balancing routine is not requested, method 1100 may proceed to 1110 to select the first batch of OCT images. For example, if the dataset includes at least a first threshold number of OCT images, then method 1100 may include selecting the first threshold number of OCT images. The selection may be performed arbitrarily, so as not to bias neural network training. Alternatively, in some examples, the entire dataset may constitute the first batch of OCT images, and no selection process is employed.

Once the first batch of OCT images has been selected (e.g., at 1108 or at 1110), method 1100 may proceed to 1112 to perform data augmentation on the first batch of OCT images. In general, data augmentation routines are employed to add features to a dataset (e.g., in preparation for neural network training) without collecting new data. In object detection applications, simple image processing techniques, such as transformations, rotations, reflections, and color alterations may be applied to images in the dataset to improve identification of desired objects. As such, data augmentation may provide an increased number of OCT images in the dataset without further input from medical professionals, further OCT scans, etc. It will be appreciated that numerous data augmentation routines are well-known to those skilled in the art and will therefore be referenced only generally herein as relates to the described embodiments. As noted above, in alternative examples, rotational invariance and robustness to noise may be included in some embodiments of the present disclosure, such that no data augmentation is employed.

At 1114, after the first batch of OCT images has been selected and preprocessed, method 1100 may include training the neural network with the first batch of OCT images, where each positive image therein may include indications of one or more provisional TCFA regions. In this way, training of the neural network may be based upon the selected first batch of OCT images which indicate medical opinions of one or more medical professionals as to a presence or an absence of TCFAs.

Specifically, at 1116, method 1100 may include identifying, via the neural network, one or more image features in the first batch of OCT images. The identifying may occur via a deep learning module, such as deep learning module 210, as described above with reference to FIG. 2. In some examples, the one or more image features may include one or more TCFA features (e.g., image features corresponding to one or more TCFAs). As an example, the neural network may identify one or more image features indicating a TCFA in a subset of OCT images, where all other remaining OCT images (e.g., apart from the subset) are identified as including no TCFA. In some examples, the neural network may identify one or more additional image features depicting one or more additional TCFAs in the subset of OCT images. In one example, the neural network may identify one or more additional image features depicting one or more additional TCFAs in a single OCT image. In this way, the neural network may be trained to generally identify TCFAs in OCT images.

At 1118, method 1100 may include determining whether a region of interest batch balancing routine is requested. If the region of interest batch balancing routine is requested, method 1100 may proceed to 1120 to set a second balancing ratio. The second balancing ratio may be a desired ratio of dominant class members to minority class members, that is, of positive images to negative images, for neural network training. The second balancing ratio may be predetermined to minimize spurious characterizations of TCFA regions (or to minimize instances of missing TCFA regions) during bounding box generation. In one example, the second balancing ratio may be 90:10. In another example, the second balancing ratio may be 80:20. In yet another example, the second balancing ratio may be 70:30. In yet another example, the second balancing ratio may be 60:40. In yet another example, the second balancing ratio may be 50:50.

At 1122, method 1100 may include selecting a second batch, or region of interest batch, of OCT images based on the second balancing ratio. For example, if the dataset includes at least 100 OCT images, and the second balancing ratio is set at 90:10, then method 1100 may include selecting 90 positive images and 10 negative images for bounding box generation. The selection may be performed arbitrarily, so as not to bias the bounding box generation routine.

Returning to 1118, if the region of interest batch balancing routine is not requested, method 1100 may proceed to 1124 to select the second batch of OCT images. For example, if the dataset includes at least a second threshold number of OCT images, then method 1100 may include selecting the second threshold number of OCT images. The selection may be performed arbitrarily, so as not to bias bounding box generation. Alternatively, in some examples, the entire first batch may constitute the second batch of OCT images, and no selection process is employed.

Once the second batch of OCT images has been selected (e.g., at 1122 or at 1124), method 1100 may proceed to 1126 to generate one or more bounding boxes based on the one or more image features identified at 1116 for those OCT images included in the second batch of OCT images. The generating may occur via a bounding boxes module, such as bounding boxes module 212, as described above with reference to FIG. 2. The neural network may segment each of the one or more OCT images into one or more regions based on the one or more image features. In examples wherein a TCFA is identified in the one or more OCT images (e.g., the subset of OCT images depicting the TCFA), the one or more regions may correspondingly include one or more first regions each depicting the TCFA. In examples wherein one or more additional TCFAs are identified in the one or more OCT images (e.g., the subset of OCT images depicting the TCFA), the one or more regions may include one or more second regions depicting the one or more additional TCFAs. In examples wherein one or more healthy portions of the artery are identified in the one or more OCT images, the one or more regions may correspondingly include one or more third regions depicting the one or more healthy portions of the artery. In examples wherein one or more low-light backgrounds are identified in the one or more OCT images, the one or more regions may correspondingly include one or more fourth regions depicting the one or more low-light backgrounds. Once the one or more OCT images is segmented into the one or more regions, the one or more bounding boxes may be generated based on the one or more regions. As such, the one or more regions may be respectively bound by the one or more bounding boxes. Thus, for example, a bounding box may be generated based on one of the one or more first regions such that the bounding box may circumscribe the TCFA indicated by the one or more TCFA features. In this way, the neural network may be trained to identify a TCFA and then localize the TCFA within an OCT image by generating a bounding box to circumscribe the TCFA. Method 1100 may then end.

In this way, an OCT imaging system including a neural network is provided, where the neural network may be trained for identifying TCFAs in OCT images. The neural network may include a trained two-stage object detection algorithm for identifying one or more OCT image features corresponding to a TCFA and generating a bounding box containing the TCFA. A technical effect of implementing the trained neural network in the OCT imaging system is that TCFAs may be automatically and accurately identified in OCT images. Such automated identification may result in improved medical diagnoses and treatments.

In one example, a method for a trained neural network, the method comprising acquiring an optical coherence tomography (OCT) image slice of an artery, identifying one or more image features of the OCT image slice with the trained neural network, and responsive to the one or more image features indicating a thin-cap fibroatheroma (TCFA), segmenting the OCT image slice into a plurality of regions with the trained neural network, the plurality of regions including a first region depicting the TCFA, and determining start and end coordinates for the TCFA based on the first region. A first example of the method further including wherein the plurality of regions further includes one or more second regions, the one or more second regions respectively depicting one or more healthy portions of the artery. A second example of the method, optionally including the first example of the method, further including wherein the plurality of regions further includes one or more third regions, the one or more third regions respectively depicting one or more additional TCFAs. A third example of the method, optionally including one or more of the first and second examples of the method, further including wherein the OCT image slice is one of a series of OCT image slices, where the series of OCT image slices is sequentially ordered. A fourth example of the method, optionally including one or more of the first through third examples of the method, further comprising acquiring remaining OCT image slices in the series of OCT image slices, identifying, for each of the remaining OCT image slices, one or more additional image features with the trained neural network, and responsive to the one or more additional image features indicating the TCFA in a subset of the remaining OCT image slices, segmenting the subset of the remaining OCT image slices into an additional plurality of regions with the trained neural network, the additional plurality of regions including one or more additional first regions, each of the one or more additional first regions depicting the TCFA, and determining, for each remaining OCT image slice in the subset of the remaining OCT image slices, additional start and end coordinates for the TCFA based on the one or more additional first regions. A fifth example of the method, optionally including one or more of the first through fourth examples of the method, further including wherein the additional plurality of regions further includes one or more third regions, the one or more third regions depicting one or more additional TCFAs. A sixth example of the method, optionally including one or more of the first through fifth examples of the method, further including wherein the first region is bounded by a rectangular bounding box. A seventh example of the method, optionally including one or more of the first through sixth examples of the method, further including wherein identifying the TCFA start and end coordinates includes determining coordinates along each of two opposite sides of the rectangular bounding box, the two opposite sides being perpendicular to a length of the TCFA in polar coordinates.

In another example, a method comprises training a neural network to identify a thin-cap fibroatheroma (TCFA) in optical coherence tomography (OCT) image slices, where identifying the TCFA includes identifying TCFA features in the OCT image slices, and generating bounding boxes in the OCT image slices for the TCFA based on the TCFA features, receiving a particular OCT image slice depicting a particular TCFA, and identifying the particular TCFA in the particular OCT image slice using the trained neural network. A first example of the method further including wherein the neural network is a convolutional neural network. A second example of the method, optionally including the first example of the method, further comprising receiving a dataset including training OCT image slices, each of the training OCT image slices including one or more provisional TCFA regions, wherein the neural network is trained based on the received dataset. A third example of the method, optionally including one or more of the first and second examples of the method, further including wherein the one or more provisional TCFA regions are respectively received from one or more medical professionals.

In yet another example, a medical imaging system comprises a scanner operable to collect optical coherence tomography (OCT) imaging data of a plaque, a memory storing a trained neural network configured to separate visual characteristics from content of an image, and a processor communicably coupled to the scanner and the memory, wherein the processor is configured to receive the OCT imaging data from the scanner, generate a sequentially ordered set of OCT images from the OCT imaging data, where a subset of the OCT images depicts the plaque, identify, via the trained neural network, the subset of OCT images depicting the plaque, generate, via the trained neural network, a bounding box circumscribing the plaque in each OCT image in the subset of OCT images, and determine, for each OCT image in the subset of OCT images, start and end coordinates for the plaque based on the bounding box. A first example of the medical imaging system further including wherein the OCT imaging data includes 3D volumetric imaging data, and the sequentially ordered set of OCT images includes 2D image slices of the 3D volumetric imaging data. A second example of the medical imaging system, optionally including the first example of the medical imaging system, further including wherein the plaque is a thin-cap fibroatheroma. A third example of the medical imaging system, optionally including one or more of the first and second examples of the medical imaging system, further comprises a display device communicably coupled to the processor, the display device including a display area, wherein the processor is further configured to include, for each OCT image in the subset of OCT images, visual indicators at the start and end coordinates, and display, via the display area of the display device, the subset of OCT images including the visual indicators. A fourth example of the medical imaging system, optionally including one or more of the first through third examples of the medical imaging system, further includes wherein identifying the subset of OCT images depicting the plaque includes identifying a series of OCT images, the series of OCT images including at least five sequential OCT images depicting the plaque, and adding the series of OCT images to the subset of OCT images. A fifth example of the medical imaging system, optionally including one or more of the first through fourth examples of the medical imaging system, further includes wherein the processor is further configured to identify a series of OCT images in the sequentially ordered set of OCT images, the series of OCT images including at least five sequential OCT images, wherein at least a first OCT image and a last OCT image are identified as including the plaque, and only one remaining OCT image is indicated as including no plaque, and indicate the plaque in each OCT image in the series of OCT images. A sixth example of the medical imaging system, optionally including one or more of the first through fifth examples of the medical imaging system, wherein the processor is further configured to identify a series of OCT images in the sequentially ordered set of OCT images, the series of OCT images including a sequential ordering of a first OCT image, a second OCT image, and a third OCT image, where the second OCT image is identified as including the plaque, and the first OCT image and the third OCT image are identified as including no plaque, and indicate no plaque in each OCT image in the series of OCT images. A seventh example of the medical imaging system, optionally including one or more of the first through sixth examples of the medical imaging system, further includes wherein the start and end coordinates are polar coordinates.

The following claims particularly point out certain combinations and sub-combinations regarded as novel and non-obvious. These claims may refer to “an” element or “a first” element or the equivalent thereof. Such claims should be understood to include incorporation of one or more such elements, neither requiring nor excluding two or more such elements. Other combinations and sub-combinations of the disclosed features, functions, elements, and/or properties may be claimed through amendment of the present claims or through presentation of new claims in this or a related application. Such claims, whether broader, narrower, equal, or different in scope to the original claims, also are regarded as included within the subject matter of the present disclosure.

1. A method for a trained neural network, the method comprising:
acquiring an optical coherence tomography (OCT) image slice of an artery;
identifying one or more image features of the OCT image slice with the trained neural network; and
responsive to the one or more image features indicating a thin-cap fibroatheroma (TCFA):
segmenting the OCT image slice into a plurality of regions with the trained neural network, the plurality of regions including a first region depicting the TCFA, and
determining start and end coordinates for the TCFA based on the first region.

2. The method of claim 1, wherein the plurality of regions further includes one or more second regions, the one or more second regions respectively depicting one or more healthy portions of the artery.

3. The method of claim 1, wherein the plurality of regions further includes one or more third regions, the one or more third regions respectively depicting one or more additional TCFAs.

4. The method of claim 1, wherein the OCT image slice is one of a series of OCT image slices, where the series of OCT image slices is sequentially ordered.

5. The method of claim 4, further comprising:
acquiring remaining OCT image slices in the series of OCT image slices;
identifying, for each of the remaining OCT image slices, one or more additional image features with the trained neural network; and
responsive to the one or more additional image features indicating the TCFA in a subset of the remaining OCT image slices:
segmenting the subset of the remaining OCT image slices into an additional plurality of regions with the trained neural network, the additional plurality of regions including one or more additional first regions, each of the one or more additional first regions depicting the TCFA, and
determining, for each remaining OCT image slice in the subset of the remaining OCT image slices, additional start and end coordinates for the TCFA based on the one or more additional first regions.

6. The method of claim 5, wherein the additional plurality of regions further includes one or more third regions, the one or more third regions depicting one or more additional TCFAs.

7. The method of claim 1, wherein the first region is bounded by a rectangular bounding box.

8. The method of claim 7, wherein identifying the TCFA start and end coordinates includes:
determining coordinates along each of two opposite sides of the rectangular bounding box, the two opposite sides being perpendicular to a length of the TCFA in polar coordinates.

9. A method, comprising:
training a neural network to identify a thin-cap fibroatheroma (TCFA) in optical coherence tomography (OCT) image slices, where identifying the TCFA includes:
identifying TCFA features in the OCT image slices; and
generating bounding boxes in the OCT image slices for the TCFA based on the TCFA features;
receiving a particular OCT image slice depicting a particular TCFA; and
identifying the particular TCFA in the particular OCT image slice using the trained neural network.

10. The method of claim 9, wherein the neural network is a convolutional neural network.

11. The method of claim 9, further comprising:
receiving a dataset including training OCT image slices, each of the training OCT image slices including one or more provisional TCFA regions,
wherein the neural network is trained based on the received dataset.

12. The method of claim 11, wherein the one or more provisional TCFA regions are respectively received from one or more medical professionals.

13. A medical imaging system, comprising:
a scanner operable to collect optical coherence tomography (OCT) imaging data of a plaque;
a memory storing a trained neural network configured to separate visual characteristics from content of an image; and
a processor communicably coupled to the scanner and the memory,
wherein the processor is configured to:
receive the OCT imaging data from the scanner;
generate a sequentially ordered set of OCT images from the OCT imaging data, where a subset of the OCT images depicts the plaque;
identify, via the trained neural network, the subset of OCT images depicting the plaque;
generate, via the trained neural network, a bounding box circumscribing the plaque in each OCT image in the subset of OCT images; and
determine, for each OCT image in the subset of OCT images, start and end coordinates for the plaque based on the bounding box.

14. The medical imaging system of claim 13, wherein
the OCT imaging data includes 3D volumetric imaging data; and
the sequentially ordered set of OCT images includes 2D image slices of the 3D volumetric imaging data.

15. The medical imaging system of claim 13, wherein the plaque is a thin-cap fibroatheroma.

16. The medical imaging system of claim 13, further comprising:
a display device communicably coupled to the processor, the display device including a display area,
wherein the processor is further configured to:
include, for each OCT image in the subset of OCT images, visual indicators at the start and end coordinates; and
display, via the display area of the display device, the subset of OCT images including the visual indicators.

17. The medical imaging system of claim 13, wherein identifying the subset of OCT images depicting the plaque includes:
identifying a series of OCT images, the series of OCT images including at least five sequential OCT images depicting the plaque; and
adding the series of OCT images to the subset of OCT images.

18. The medical imaging system of claim 13, wherein the processor is further configured to:
identify a series of OCT images in the sequentially ordered set of OCT images, the series of OCT images including at least five sequential OCT images, wherein at least a first OCT image and a last OCT image are identified as including the plaque, and only one remaining OCT image is indicated as including no plaque; and
indicate the plaque in each OCT image in the series of OCT images.

19. The medical imaging system of claim 13, wherein the processor is further configured to:
identify a series of OCT images in the sequentially ordered set of OCT images, the series of OCT images including a sequential ordering of a first OCT image, a second OCT image, and a third OCT image, where the second OCT image is identified as including the plaque, and the first OCT image and the third OCT image are identified as including no plaque; and
indicate no plaque in each OCT image in the series of OCT images.

20. The medical imaging system of claim 13, wherein the start and end coordinates are polar coordinates.
